(19) United States
(12) Patent Application Publication
(10) Pub. No.: US 2020/0397274 A1
(43) Pub. Date: Dec. 24, 2020

(54) BLADE FOR A VIDEO LARYNGOSCOPE WITH EXTENDABLE TIP

(71) Applicant: Héctor De Jesús VELEZ RIVERA, Jalisco (MX)

(72) Inventor: Héctor De Jesús VELEZ RIVERA, Jalisco (MX)

(21) Appl. No.: 16/754,884

(22) PCT Filed: Oct. 9, 2017
(86) PCT No.: PCT/IB2017/056208

§ 371 Date: Sep. 4, 2020

(51) Int. Cl. A61B 1/267; A61B 1/05; A61B 1/015; A61M 16/04

(52) U.S. Cl. CPC A61B 1/267; A61M 16/0488; A61B 1/015; A61B 1/05

(57) ABSTRACT

It is disclosed a blade of videolaryngoscope comprising an enlarged and curved main body with a proximal end and a distal end, the main body including an inner conduit running from the proximal end to the distal end, the inner conduit having a distal outlet for receiving a video camera; a tip slidably assembled into the main body, wherein the tip has a retracted position and at least one extended position with respect to the main body, a position selector is mounted to the main body and is connected to the tip; and a flexible element which due its flexibility also functions as an auto-adjustable guide for receiving endotracheal cannulas or tubes of different diameters.

### FIELD OF THE INVENTION

The instant invention refers to the techniques used in the design and construction of apparatus and devices for medical use and, more particularly, it is related to a blade of videolaryngoscope having a tip being extendable and which allows the use of the videolaryngoscope in patients with different anthropometric proportions in the neck, the blade further comprises a flexible element for avoiding injuries to the patient's tooth or upper gum when the blade of the videolaryngoscope is inserted into the patient's mouth. Due its flexibility, the flexible element also functions as an auto-adjustable guide for receiving endotracheal cannulas or tubes of different diameters.

### BACKGROUND OF THE INVENTION

The laryngoscope is an instrument used for directly visualizing the larynx in order to perform an endotracheal intubation for delivering anesthetics. The laryngoscope basically comprises two main portions, specifically, a handle for handling the laryngoscope and a blade assembled into the handle which is inserted into the larynx of the patient and it functions to separate the tongue and the epiglottis. At the end of the blade, usually there is a light source (a small light bulb or a light point of optical fiber originating in the handle). In the case of the videolaryngoscopes, a camera is embodied at the end of the blade in order to capture images to be displayed and a lighting system is provided.

The blade may be reusable, in which case the same must be sterilized after each use or the blade may be disposable. The blades are provided with channels or conduits for the passage of an endotracheal cannula or tube.

In the prior art, various types of blades can be found, being one of the main and most widely used the Macintosh blade, which is a single piece and has a parabolic curved path with the distal third or the tip having a straight path.

Notwithstanding, although a wide variety of blades have been developed for laryngoscopes, there are medical requirements and problems which have not been sufficiently addressed by the existing techniques, one of them related to the fact that the length of the blade is not sufficient to reach a required area in the larynx, this particularly happens in patients with long necks, where additionally the diameters of the endotracheal cannula do not always adjust on the blade to adequately perform the intubation procedure, that is, depending on the anthropometry of the patient, sometimes it is required to use endotracheal cannulas of a larger diameter or a smaller diameter but using the same blade length or vice versa, that is, the blade length is correct but intubation requires some particular type of endotracheal cannula or tube.

Now then, another problem produced with the blades of laryngoscope is that they have hard and heavy parts due they are manufactured of stainless steel, so they can provoke accidents mainly to the upper tooth of patients, it is not unusual a situation in which a dental piece of the upper gum falls or damages when contacting the blade due the handling of the laryngoscope.

In the state of the art, there are some laryngoscopes or blades containing flexible or movable elements such the laryngoscope of document CN202161249U or the laryngoscope with flexible blade of document EP944346, however, such documents do not solve the aforementioned problems.

Currently, the inclusion of cameras and lights in the laryngoscopes has gave origins to videolaryngoscopes which allow the visualization of the larynx in a display associated to the laryngoscope, the display may be assembled into the handle of the laryngoscope or assembled into a monitor operatively communicated to the camera.

### SUMMARY OF THE INVENTION

In order to overcome the problems of the state of the art, a blade of videolaryngoscope having an extendable tip has been developed, particularly, the blade of the present invention comprises an enlarged and curved main body with a proximal end and a distal end, the main body includes an inner conduit running from the proximal end to the distal end, the inner conduit having a distal outlet which receives a video camera being part of the videolaryngoscope in which the blade of present invention is assembled.

Another element of present invention is a tip slidably mounted to the main body, wherein the tip has a retracted position and at least one extended position with respect to the main body in order to have different lengths with respect to the main body and, therefore, having different useful positions of the blade for performing the required intubation.

Likewise, the blade of laryngoscope comprises a position selector mounted to the main body and connected to the slidable tip. Finally, the blade of the laryngoscope comprises a flexible element including a tube or endotracheal cannula guide, being the flexible element attached below the inner conduit.

In the preferred embodiment of present invention, for slidably connecting the tip to the main body, the body includes a sliding slot running at a distal portion of the main body while the tip includes a sliding portion running inside and along said sliding slot for a movement between the retracted position and the at least one extended position.

Preferably, the position selector comprising a lateral wall that is integrally provided at a side of the sliding section of the tip and protruding downwards the same; a button positioned in such lateral wall and laterally protruding thereof, and a cover having a longitudinal slot provided with at least two notches located along the conduit, wherein the button moves between said notches along the longitudinal slot, particularly, the cover is mounted into the main body, such that when pushing down the button and moving it through the longitudinal slot, the tip is released and it may be extended or retracted with respect the main body until the button engages once again one of the notches provided in the longitudinal slot.

On the other hand, the flexible element is made of an elastomer and it comprises a bottom wall below attached to the inner conduit; a lateral wall attached to a side of the inner conduit; a rising rim separated from the lateral wall such that between the space formed by such lateral wall and the rising rim a longitudinally running endotracheal cannula or tube is received. Due the flexibility of the flexible element, the same also functions as an auto-adjustable guide for receiving endotracheal cannulas or tubes of different diameter.

In another embodiment of the invention, the width of the flexible element is greater than the width of the main body in order to form a protection surface for avoiding damages and losses of the dental pieces. Furthermore, the flexible element may rest on the gum, or it may be adjusted in the free space of lost front tooth by the patient, that is, in case the patient has not front tooth, the flexible element is allocated in such free space.

Additionally, the tip includes a reinforcing wall with a decreasing height in distal direction satisfying two objectives, one is providing resistance to the tip and the other is providing a free view of the camera once the blade is inserted into the patient due the decreasing height.

### BRIEF DESCRIPTION OF THE DRAWINGS

The novel aspects which are characterizing of present invention will be established, particularly, in the attached claims. However, the invention together with other objectives and advantages thereof should be better understood in the below detailed description of some preferred embodiments of the invention, when reading together with the figures, which disclose:

FIG. 1 is a front and right lateral perspective of a blade of videolaryngoscope constructed according the principles of present invention.

FIG. 2 is a front and left lateral perspective of the blade of videolaryngoscope of FIG. 1.

FIG. 3 is an exploded view of the blade of videolaryngoscope of FIG. 1.

FIG. 4 is a left lateral view of the blade of videolaryngoscope with its tip in a retracted position.

FIG. 5 is a right lateral view of the blade of videolaryngoscope with its tip in the same position that FIG. 4.

FIG. 6 is a left lateral view of the blade of videolaryngoscope with its tip in an extended position.

FIG. 7 is right lateral view of the blade of videolaryngoscope with its tip in the same position that FIG. 6.

### DETAILED DESCRIPTION OF THE PREFERRED EMBODIMENTS OF THE INVENTION

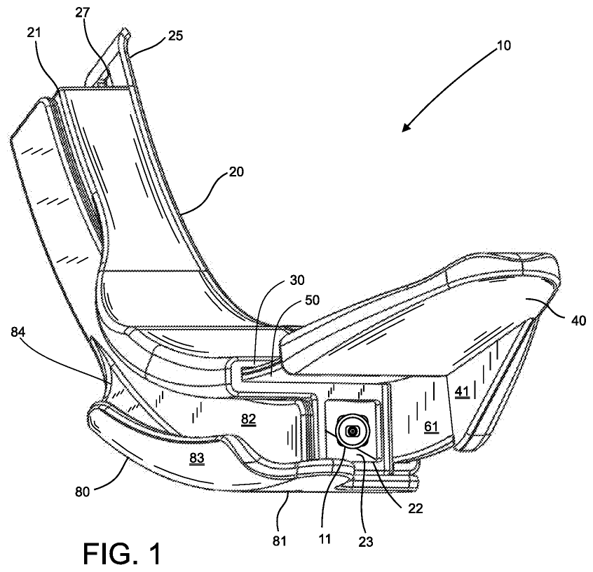

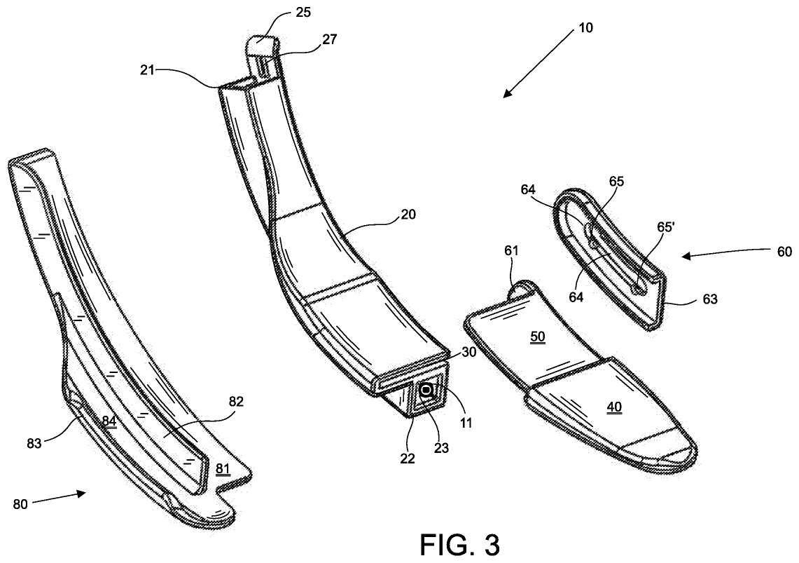

Referring to FIGS. 1 to 3 of the attached figures, it is shown a blade of videolaryngoscope 10 constructed according to the principles of present invention, this embodiment must be only considered as illustrative but not limitative of the invention.

In FIGS. 1 to 3, it is appreciable that the blade 10 comprises an enlarged and curved main body 20 with a proximal end 21 and a distal end 22, the main body 20 includes an inner conduit 23 running from the proximal end 21 to the distal end 22, said inner conduit 23 has a quadrangular shape in transversal section with an upper wall opposite to a lower wall and has opposite lateral walls connecting the upper wall to the lower wall. The inner conduit 23 has a distal outlet, which may receive a video camera 11 that is part of any videolaryngoscope wherein the blade of the present invention is mounted. The video camera 11 allows viewing the interior of the larynx when the blade is inside the neck of the patient, it is worth noting that the video camera is not part of the blade of present invention, but it is illustrated to note the advantages and use of the blade once assembled into a given videolaryngoscope.

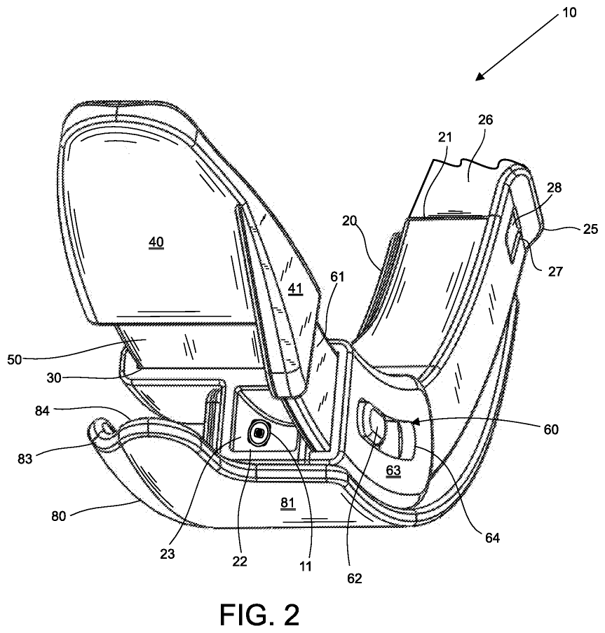

The inner conduit 23 has the main function of receiving, along its total length, a blade mounting base 26 (see FIG. 2) of a laryngoscope, such mounting base 26 is partially shown in FIG. 2, the base is coupled to the blade 10 by means of a flexible mounting portion 25 provided in the proximal end 21 of the blade 10, the proximal end includes a hole 27 which receives a protrusion 28 provided in the mounting base; another mounting forms can be conceived without departing from the scope of present invention.

Another main part of present invention is a tip 40 slidably mounted to the main body 20, wherein the tip 40 has a retracted position and at least one extended position with respect to the main body 20. This allows a videolaryngoscope including the blade 10 may be used in patients having a longer neck than others. Now, the blade 10 has a position selector 60 mounted to the main body 20 and connected to the tip 40.

Additionally, the blade 10 includes a flexible element 80 attached to the main body 20, particularly to the bottom wall, and a lateral wall of the inner conduit 23, this flexible element 80 includes an endotracheal cannula or tube guide, and allows receiving endotracheal cannulas or tubes of different diameters, said flexible element 80 is made of an elastomer of medical grade which does not provoke damages to the patients due it is a soft material, thus minimizing the risk to damage the dental pieces.

The blade of the present invention in general terms has the geometry of a Macintosh blade, which is well known by one skilled in the art, but this is not a limitation to apply the principles of present invention to other type of blades as Miller blades. For example, in the illustrated embodiment, the extendable tip 40 allows varying the length of the blade between two grades of laryngoscope blade, e.g., a Macintosh blade of grade 3 to a Macintosh blade of grade 4, or a greater grade o well an intermediate grade, which allows use the blade in patients with longer necks. Preferably, the tip is extendable between about 0.1 cm and about 10 cm.

In order to slidably mount the tip 40 with respect to the main body 20, the body includes a sliding slot 30 being narrow and running along from a distal portion of the main body 20 which is above the upper wall of the inner conduit 23, while the tip includes a sliding portion 50 which couples and runs inside such sliding slot 30 so as to slide the tip 40 with respect to the main body 20 between the retracted position and the at least one extended position. As seen, the sliding portion 50 of the tip 40 is curved in its length for running inside the sliding slot 30 and thus extending or retracting the tip 40.

The tip 40 includes a reinforcing wall 41 extending in a distal direction and having a height decreasing in the same direction in order to provide resistance to the tip and to provide a free view to the video camera. In the embodiment disclosed, such reinforcing wall 41 comprises a length smaller than the half of the tip.

As shown, the sliding slot 30 is located above the inner conduit 23 of the main body 20, such as mentioned said inner conduit has a quadrangular shape in transversal section with opposite lateral walls, an upper wall and a lower wall.

Regarding the position selector 60, this comprises a flexible lateral wall 61 integrally provided in a side of the tip 40, particularly the lateral wall 61 provided in the side of the sliding portion 50 and being perpendicular thereto; the position selector also comprises a button 62 located in said lateral wall 61. The selector also has a cover 63 with a longitudinal slot 64 provided with at least two notches 65 and 65′ located along the longitudinal slot 64, particularly notches 65 and 65′ are located at the ends of such slot, but they may be also provided in an intermediate point between the ends of the longitudinal slot 64. The cover 63 is mounted to the main body 20, wherein the button 62 is movable between such notches 65 and 65′ along the longitudinal slot. In such manner, when pushing down button 62, tip 40 is released and it may be extended or retracted with respect to the main body 20 until button 62, when sliding along the longitudinal slot 64, is once again engaged in one of the notches 65 or 65′.

As above mentioned, a problem of the state of the art is the possibility of damage of the dental pieces when the blade is inserted into the mouth in order to intubate, thus, the present invention comprises a flexible element 80 comprising a bottom wall 81 attached to the inner wall of the inner conduit 23, the bottom wall 81 is slightly curved in transversal section. Likewise, the flexible element also includes a lateral wall 82 completely joined to the lateral wall of the right side of the inner conduit 23; another portion of the flexible element is a rising rim 83 protruding from the bottom wall and being separated from lateral wall 82, wherein, between the space formed by such lateral wall 82 and the rising rim 83, a guide 84 of endotracheal cannula or tube is formed, due to this geometry and the use of a flexible material, the guide of endotracheal cannula or tube may receive tubes with different diameters, the flexible element is auto-adjustable to the tubes or cannulas due its flexibility. The flexible element is made of elastomer materials, which are well known for one, skilled in the art.

Now, the width of the flexible element 80 is greater than the width of the main body as seen in FIG. 2 whereby the bottom wall 81 provides protection to the dental pieces, also the flexible element 80 can be adjusted in the free space left by previously lost dental pieces or it may rest on the gum or the palate when the blade 10 is inserted into the mouth of the patient.

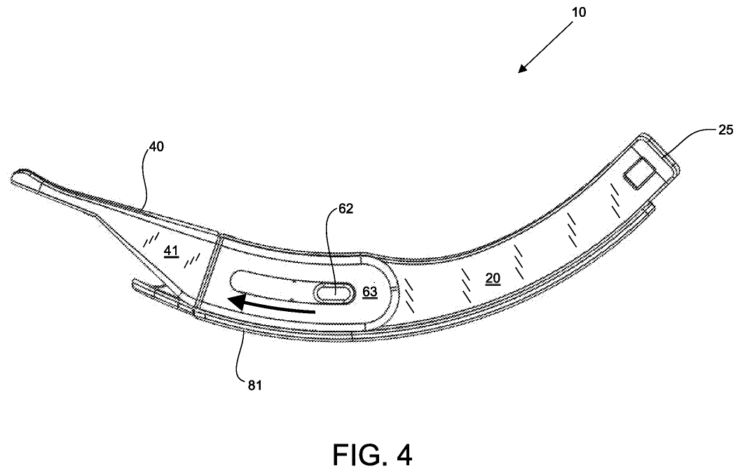

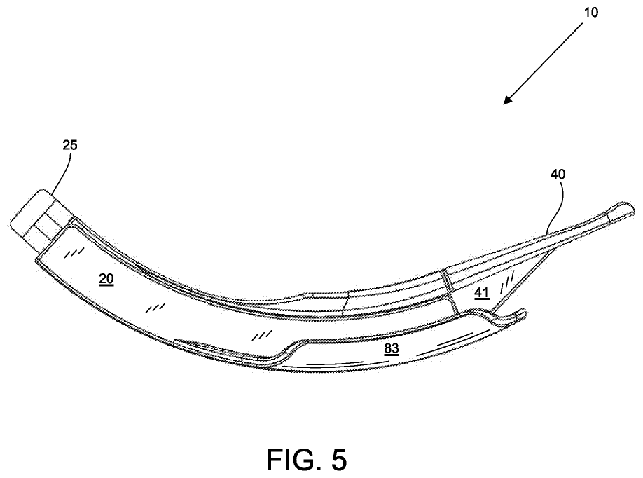

Once the configuration of the main parts of the blade of videolaryngoscope has been disclosed, reference is made to FIGS. 4 and 5 in which the blade of videolaryngoscope 10 is shown from its left and right side respectively, in such FIGS. 4 and 5 the tip 40 is in a retracted position with respect to the main body 20, whereby when pushing down the button 62 protruding from the cover 63, the button is released and when is moved to the distal direction, the tip 40 is moved with respect the main body, such FIGS. 4 and 5 also allow observing the bottom wall 81, the reinforcing wall 41 of the tip and the flexible mounting portion 25.

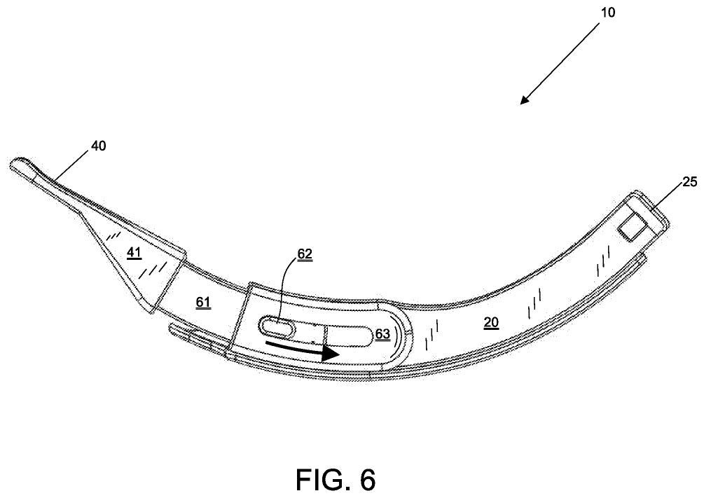

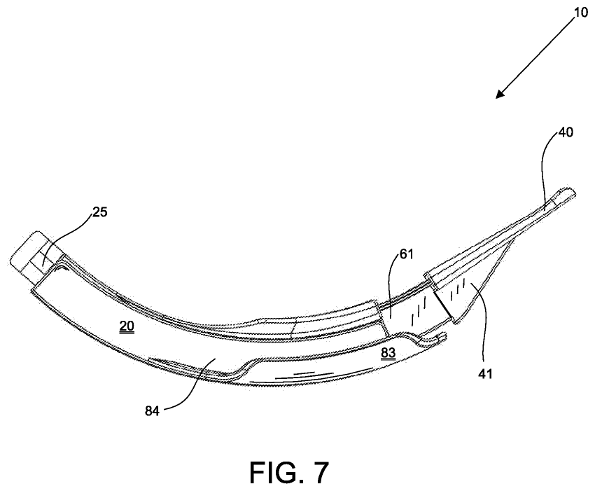

In FIGS. 6 and 7, it is shown the blade of videolaryngoscope 10 with the tip 40 in its extended position once the button 62 provided in the lateral wall 61 of the tip has been moved over cover 63 in distal direction whereby the tip 40 has been moved in distal direction with respect to the main body 20, once again and particularly in FIG. 7, it is shown the reinforcing wall 41 and the guide of endotracheal cannula o tube 84.

Finally, it is convenient to mention that the edges of the blade which are in contact with the larynx are round in order to avoid damages to the patient.

According to the previous disclosure, it is obvious for one skilled in the art that the embodiment of the blade of videolaryngoscope previously illustrated is just illustrative, since one skilled in the art may perform different variations of the same, when and if they are designed according to the principles of present invention. Accordingly, present invention includes all the embodiments that one skilled in the art may derive from the concepts contained in present description, according to the following set of claims.

### REFERENCE LIST

10 Blade of laryngoscope
11 Video camera
20 Main body
21 Proximal end
22 Distal end
23 Inner conduit
25 Mounting portion for laryngoscope
26 Blade mounting base
27 Hole
28 Protrusion
30 Sliding slot
40 Tip
41 Reinforcing wall
50 Sliding portion
60 Position selector
61 Lateral wall
62 Button
63 Cover
64 Longitudinal Slot
65, 65′ Notches
80 Flexible element
81 Bottom wall
82 Lateral wall
83 Rising rim
84 Guide of endotracheal cannula or tube.

What is claimed is:

1. A blade of videolaryngoscope comprising:
an enlarged and curved main body with a proximal end and a distal end, the main body including an inner conduit running from the proximal end to the distal end, the inner conduit having a distal outlet which receives a video camera of the video laryngoscope;
a tip slidably assembled into the main body, wherein the tip has a retracted position and at least one extended position with respect to the main body in order to have different useful lengths of the blade;
a position selector mounted into the main body and connected to the tip allowing movement of the tip between the retracted position and the at least one extended position; and
a flexible guide comprising:
a bottom wall below attached to the inner conduit;
a lateral wall attached to a side of the inner conduit; and
a rising rim separated from the lateral wall, whereby a guide for an endotracheal cannula or tube is formed between the space formed by the lateral wall and the rising rim, the flexible element forming a protection surface of dental pieces.

2. The blade of videolaryngoscope of claim 1, wherein the main body includes a sliding slot running in a distal portion of the main body and the tip includes a sliding portion that couples and runs inside said sliding slot to move between the retracted position and the at least one extended position.

3. The blade of videolaryngoscope of claim 2, wherein the sliding slot is located above the inner conduit of the main body.

4. The blade of videolaryngoscope of claim 2, wherein the sliding section of the tip has a flat width and a curved length.

5. The blade of videolaryngoscope of claim 1, wherein the position selector comprises:
a lateral wall provided at a side of the tip;
a button located on said lateral wall and laterally protrudes thereof,
a cover with a longitudinal slot provided with at least two notches located along the inner conduit, the button moving between such notches along the longitudinal slot wherein the cover is mounted to the main body, such that, when pushing down the button, the tip is released and it may be extended or retracted with respect the main body until the button engages one of the notches.

6. The blade of videolaryngoscope of claim 1, wherein the flexible material of the guide of endotracheal cannula or tube is an elastomer.

7. The blade of videolaryngoscope of claim 1, wherein the width of the flexible element is greater than the width of the main body in order to form the protection surface of dental pieces or to adjust to the separation space between tooth or to rest on the gum.

8. The blade of videolaryngoscope of claim 1, wherein tip is extendable between 0.1 and 10 cm with respect the main body.

9. The blade of videolaryngoscope of claim 1, wherein the tip includes a reinforcing wall with a decreasing height in distal direction in order to provide resistance to the tip and a free view to the camera in distal direction.

10. The blade of videolaryngoscope of claim 9, wherein said reinforcing wall has a length smaller than the half of the tip.

11. The blade of videolaryngoscope of claim 1, wherein the edges of the blade are round.

12. The blade of videolaryngoscope of claim 1, wherein the inner conduit has a quadrangular shape in transversal section with an upper wall opposite to a lower wall and opposite lateral walls.

13. The blade of videolaryngoscope of claim 1, wherein the blade comprises in its proximal end an assembly portion including a hole for receiving a shoulder of a mounting base of the videolaryngoscope.
